(19) United States
(12) Patent Application Publication
(10) Pub. No.: US 2008/0274166 A1
(43) Pub. Date: Nov. 6, 2008

(54) Patch for Transdermal Drug Delivery

(75) Inventors: Hagit Sacks, Modi'in (IL); Meir Stern, Rehovot (IL); Galit Levin, Nordiya (IL); Bar El Yossi, Beit Arie (IL); Giora Arbel, Tel Mond (IL)

Correspondence Address: DARBY & DARBY P.C., P.O. BOX 770, Church Street Station, New York, NY, 10008-0770, US

(73) Assignee: Transpharma Medical Ltd., Yehud (IL)

(21) Appl. No.: 11/915,831

(22) PCT Filed: Jun. 11, 2006
(86) PCT No.: PCT/IL2006/000679

§ 371 Date: Jan. 14, 2008

Related U.S. Application Data
(60) Provisional application No. 60/689,763, filed on Jun. 10, 2005.

(51) Int. Cl. A61K 9/70; A61P 43/00

(52) U.S. Cl. 424/449

(57) ABSTRACT

Apparatus is provided including a transdermal drug delivery patch product (10, 100), which includes a patch (20, 120), which includes a drug; and protective packaging, adapted to store the patch (20, 120), and to allow the drug to dry while the patch (20, 120) is stored in the protective packaging. Other embodiments are also described.

### CROSS-REFERENCES TO RELATED APPLICATIONS

The present application claims the benefit of U.S. Provisional Patent Application 60/689,763, filed Jun. 10, 2005, entitled, “Patch for transdermal drug delivery,” which is assigned to the assignee of the present application and is incorporated herein by reference.

### FIELD OF THE INVENTION

The present invention relates generally to techniques for drug delivery, and specifically to methods and apparatus for transdermal drug delivery.

### BACKGROUND OF THE INVENTION

Adhesive transdermal drug patches deliver a drug across the skin directly into the systemic blood circulation. Typically, the drug is dispersed in the adhesive that attaches the patch to the skin.

PCT Publication WO 03/039620 to Sohn, which is assigned to the assignee of the present application and is incorporated herein by reference, describes apparatus for facilitating delivery of a substance through skin of a subject. The apparatus includes a handle and a cartridge, removably coupled to the handle. The cartridge includes one or more electrodes and a patch comprising the substance, the electrodes adapted to be applied to a region of the skin, and the patch adapted to be applied to at least a portion of the region of the skin by removal of the electrodes therefrom. Also described is a medicated patch which comprises a lower backing, a pad, and an upper protective cover covering pad. The pad has two sections: (a) an electrical treatment side, having a shaped portion such as a frame that surrounds an open portion passing therethrough, and (b) a substance-treatment side, having a medicated region. The lower backing covers an adhesive region surrounding the open portion on the underside of the electrical treatment side (which underside comes in contact with the skin). The upper cover protects the upper portion of pad, including the substance-containing region, from contamination. The upper cover comprises a tab, connected to an edge near the open portion.

U.S. Pat. No. 5,603,693 to Frenkel et al., which is incorporated herein by reference, describes a device having three separable modules, for the transdermic administration of drugs by electrophoresis or iontophoresis, which comprises a first active module provided with at least one system of electrodes and one drug reservoir, a second power module provided with a power supply, and a third electronic module having an electronic circuit, control organs, and a display screen. The power module is situated between the two other modules and comprises, in addition to the power supply formed by one or more batteries, mechanical assembly means and electrical connection or interconnection means with the two other modules means for attaching the device to the body of a patient.

U.S. Pat. No. 5,908,401 to Henley, which is incorporated herein by reference, describes a portable iontophoresis apparatus for facilitating delivery of medication across the cutaneous membrane into adjacent underlying tissues and blood vessels. The apparatus employs a modular, detachable non-reusable medicament-containing applicator electrode which is adapted to attach to a base assembly. The apparatus is designed to be hand-held and includes a circumferential tactile electrode band on the base assembly which provides electrical connection between the skin of the user's hand and one pole of a bipolar power source housed within the base assembly. The opposing pole of the power source is connected to the applicator electrode. The user's body completes the electrical circuit between the applicator and tactile electrodes.

U.S. Pat. No. 5,919,156 to Stropkay et al., which is incorporated herein by reference, describes an iontophoretic drug delivery system including a plurality of patches, at least one reusable controller, and a unit for storing and dispensing the patches. The patches may be secured in a compartment formed in the unit and the controller may be stored in another compartment formed in the unit. In this way, the reusable controller and a new patch can be removed from the unit and fastened to one another for activation and attachment to the skin of a patient.

U.S. Pat. No. 3,163,166 to Brant et al., which is incorporated herein by reference, describes a method for the treatment by iontophoresis of a selected area of soft tissue surface, including passing an electric current between a selected area of soft tissue surface and an external electrode. The current is passed through electrolyte interposed and maintained between the external electrode and the area of soft tissue surface while keeping the electrode in motion over the area.

### SUMMARY OF THE INVENTION

In embodiments of the present invention, a transdermal drug delivery patch product comprises a patch and protective packaging for storing and protecting the patch prior to use. The protective packaging typically comprises a package, in which the patch is stored. During manufacture, the drug is applied to the patch in liquid form, such as by using a printing-like process or another technique known in the art. The drug is typically allowed to partially dry for a short time before being placed in the package. The protective packaging is configured to allow the drug to continue drying on the patch after the patch has been inserted into the package. Such post-packaging drying typically reduces manufacturing time, complexity, and/or cost. The drug generally has greater stability and shelf life when in a substantially dry form than in a liquid or only partially-dry form.

In some embodiments of the present invention, the patch comprises a backing liner having an adhesive area and a drug delivery area. The protective packaging comprises (in addition to the package) a removable protective covering applied to the patch. The protective covering is configured to substantially not come in contact with the drug delivery area of the patch, and is shaped so as to define one or more holes therethrough. The package stores the patch together with the protective covering prior to use. The package typically comprises a moisture-absorbing desiccant, such as a silica gel desiccant. The desiccant is in fluid communication, via the holes, with the drug delivery area of the patch. The desiccant thus absorbs moisture from the drug while it is in its partially-dry form, thereby completing the drying of the drug to a level of dryness suitable for long-term storage.

It is noted that different drugs typically have different moisture content levels in the partially-dry form, and also have different moisture content levels that are suitable for long-term storage. Nevertheless, by way of illustration and not limitation, some embodiments of the invention include reducing the moisture content to a partially-dry level of between about 6% and 10% or between about 10% and 15% by weight before placing the patch into the package. Alternatively or additionally, embodiments of the invention include reducing the moisture content from the partially-dry level to between about 2% and 3% or between about 3% and 4% or between about 4% and 5%, after the patch has been placed in the package.

Typically, the protective covering is held to the patch by the adhesive area, which is also used to attach the patch to the skin.

There is therefore provided, in accordance with an embodiment of the present invention, apparatus including a transdermal drug delivery patch product, which includes:

a patch, which includes a drug; and

protective packaging, adapted to store the patch, and to allow the drug to dry while the patch is stored in the protective packaging.

For some applications, the protective packaging is configured to dry the drug to a moisture content of 2-3% or 3-5% by weight while the patch is stored in the protective packaging.

In an embodiment, the patch is shaped so as to define: a first portion shaped so as to define a frame that surrounds a window, and a second portion, adjacent to the first portion, that defines a drug delivery area including the drug, and the patch is configured such that when the second portion is folded onto the first portion, the drug delivery area is placed through the window.

In an embodiment, the protective packaging includes a package, adapted to store the patch, and the package includes a desiccant in fluid communication with the drug when the patch is stored in the package.

In an embodiment, the protective packaging includes a removable protective covering applied to the patch, configured such that during storage, when the protective covering is applied to the patch, at least a portion of the protective covering is spaced away from the drug, and the protective covering is shaped so as to define one or more holes therethrough. For some applications, the protective packaging includes a package, adapted to store the patch together with the protective covering, and including a desiccant in fluid communication with the drug via the holes when the patch is stored in the package. For some applications, the protective covering includes exactly one element. For some applications, the element is shaped so as to define a peripheral area, and a raised central area shaped so as not to come in contact with the drug.

In an embodiment, the protective packaging includes a removable protective covering applied to the patch, configured such that during storage, when the protective covering is applied to the patch, at least a portion of the protective covering is spaced away from the drug, and the protective covering includes a desiccant. For some applications, the desiccant is disposed on a face of the protective covering that faces the patch. Alternatively, for some applications, the protective covering is shaped so as to define one or more holes therethrough, and the desiccant is disposed on a face of the protective covering that faces away from the patch, such that the desiccant is in fluid communication with the drug via the holes when the patch is stored in the protective packaging.

For some applications, the protective covering is shaped so as to define between 1 and 10 holes therethrough, between 10 and 100 holes therethrough, or greater than 100 holes therethrough.

For some applications, the protective covering includes at least one item selected from the list consisting of: a cloth, a plastic, a screen, a mesh, a porous material, and a moisture-permeable film, and the one or more holes are holes in the selected item.

There is further provided, in accordance with an embodiment of the present invention, a method for manufacturing a transdermal drug delivery patch product, the method including:

applying a drug to a patch; and

while at least a portion of the drug has greater than 6% moisture content by weight, storing the patch in protective packaging adapted to dry the drug while the patch is stored in the protective packaging.

In an embodiment, storing the patch in the protective packaging includes placing a desiccant in the protective packaging.

For some applications, storing the patch includes storing the patch in the protective packaging, the protective packaging being adapted to dry the drug to a moisture content of 2-6% or 3-5% by weight while the patch is stored in the protective packaging.

In an embodiment, the protective packaging includes a package which includes a desiccant, and storing the patch includes storing the patch in the package such that the desiccant is in fluid communication with the drug. For some applications, the protective packaging includes a removable protective covering shaped so as to define one or more holes therethrough, and storing the patch includes applying the protective covering to the patch such that at least a portion of the protective covering is spaced away from the drug. Alternatively, for some applications, the protective packaging includes a package which includes a desiccant, and storing the patch includes storing the patch in the package together with the protective covering such that the desiccant is in fluid communication with the drug via the holes.

For some applications, the protective packaging includes a removable protective covering including a desiccant, and storing the patch includes applying the protective covering to the patch such that at least a portion of the protective covering is spaced away from the drug. For some applications, the desiccant is disposed on a face of the protective covering that faces the patch. For some applications, the protective covering is shaped so as to define one or more holes therethrough, the desiccant is disposed on a face of the protective covering that faces away from the patch, and storing the patch includes applying the protective covering to the patch such that the desiccant is in fluid communication with the drug via the holes.

There is still further provided, in accordance with an embodiment of the present invention, a method for transderrnally administering a drug to a subject, including:

applying a drug to a patch;

while at least a portion of the drug has greater than 6% moisture content by weight, storing the patch in protective packaging;

allowing the drug to dry while the patch is stored in the package; and

after the drug dries, applying the patch to skin of the subject, such that moisture from the skin dissolves the drug.

In an embodiment, the patch is shaped so as define a first portion and a second portion adjacent to the first portion, and applying the patch to the skin includes:

adhering a frame of the first portion to the skin such that a portion of the skin is exposed through a window defined by the frame; and

folding the second portion onto the first portion, such that a drug delivery area of the second portion that includes the drug is applied through the window to the portion of the skin.

In an embodiment, storing the patch in the protective packaging includes placing a desiccant in the protective packaging.

In an embodiment, the protective packaging includes a removable protective covering shaped so as to define one or more holes therethrough, storing the patch includes applying the protective covering to the patch such that at least a portion of the protective covering is spaced away from the drug, and applying the patch includes removing the protective covering from the patch.

In an embodiment, the protective packaging includes a package which includes a desiccant, and storing the patch includes storing the patch in the package together with the protective covering such that the desiccant is in fluid communication with the drug via the holes.

The present invention will be more fully understood from the following detailed description of embodiments thereof, taken together with the drawings, in which:

### BRIEF DESCRIPTION OF THE DRAWINGS

FIGS. 1A-D are schematic illustrations of a transdermal drug delivery product, in accordance with an embodiment of the present invention;

FIGS. 2A-C are schematic illustrations of another transdermal drug delivery product, in accordance with an embodiment of the present invention;

FIGS. 3A-D are schematic illustrations of application of a patch of the product of FIGS. 1A-D or FIGS. 2A-C to skin of a subject, in accordance with an embodiment of the present invention;

FIGS. 4, 5, and 6A-B are schematic illustrations of yet another transdermal drug delivery product, in accordance with an embodiment of the present invention; and

FIGS. 7A-E are schematic illustrations of application of a patch of the product of FIGS. 4, 5, and 6A-B to skin of a subject, in accordance with an embodiment of the present invention.

### DETAILED DESCRIPTION OF EMBODIMENTS

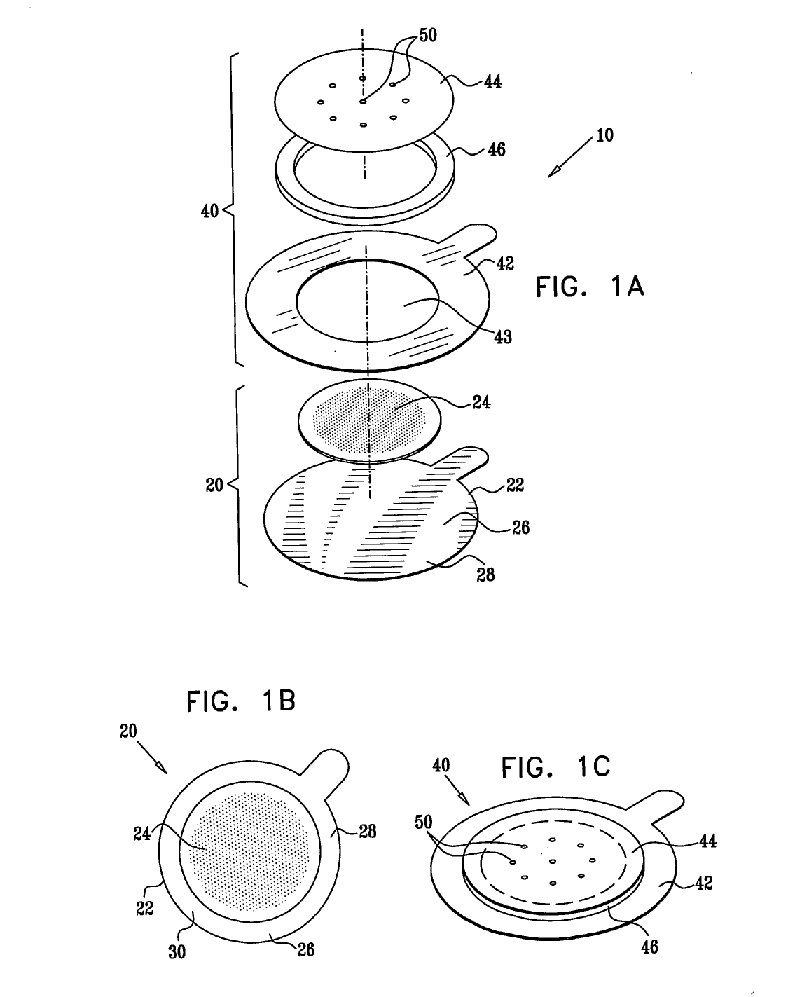

FIGS. 1A-D are schematic illustrations of a transdermal drug delivery product 10, in accordance with an embodiment of the present invention. FIG. 1A shows a portion of the elements of product 10 prior to assembly. Product 10 comprises a patch 20, which typically comprises a backing liner 22 and a drug delivery area 24. A surface 26 of backing liner 22 typically comprises an adhesive 28. As shown in FIG. 1B, a portion of adhesive 28 typically secures drug delivery area 24 to surface 26, while the remainder of adhesive 28 remains exposed and defines an adhesive area 30, typically around the periphery of backing liner 22. Adhesive area 30 typically serves both to secure patch 20 to a removable protective covering 40, described hereinbelow, and to secure the patch to skin of a subject after the protective covering has been removed. The scope of the invention includes functionally equivalent alternative configurations of patch 20, such as forming drug delivery area 24 as an integrated component of backing liner 22, e.g., by applying a drug directly to a portion of backing liner 22.

Reference is made to FIGS. 1A and 1C, which show the elements of protective covering 40 unassembled and assembled, respectively. Protective covering 40 is configured to substantially not come in contact with drug delivery area 24 when the protective covering is applied to patch 20. For some applications, protective covering 40 comprises: (a) a lower layer 42, which is brought into contact with adhesive area 30 of backing liner 22, and which typically defines a central opening 43 therethrough, (b) an upper layer 44, which provides protection for drug delivery area 24 from mechanical contact with the environment, and (c) a spacing layer 46, which prevents upper layer 44 from coming in contact with drug delivery area 24 or minimizes the likelihood of such contact under normal handling conditions. For example, a plurality of transdermal drug delivery products 10 may be stored in a box intended for retail sale, such that the box prevents undue forces from being applied to each package 60, as described hereinbelow with reference to FIG. 1D (and thereby minimizes the likelihood of contact between upper layer 44 and drug delivery area 24 in each package). The scope of the invention includes functionally equivalent alternative configurations of protective covering 40. For example, lower layer 42 and upper layer 44 may comprise a single protective layer, and spacing layer 46 may be coupled to a lower surface of the single protective layer, so as to prevent contact between a central portion of the single protective layer and drug delivery area 24 (configuration not shown).

Upper layer 44 of protective covering 40 is typically configured to allow gas passage therethrough. Typically, upper layer 44 is shaped so as to define one or more holes 50 therethrough for this purpose. The gas within package 60 is typically inert, and may comprise, as appropriate, nitrogen and/or argon. Alternatively, the gas comprises air or another combination of gases.

Reference is made to FIG. 1D, which shows the complete product 10 after assembly, including patch 20 and protective covering 40, as described hereinabove, and a package 60 for storing patch 20 together with protective covering 40. Package 60 typically comprises a moisture-absorbing desiccant 62, such as a silica gel desiccant. For example, desiccant 62 may comprise a conventional desiccant bag or other desiccant container typically used in drug packaging. For some applications, about 0.25 g to about 3 g of desiccant 62 is provided (e.g., about 0.5 g to about 2 g). Desiccant 62 may be fixed to or removable from package 60, as appropriate. Alternatively or additionally, desiccant 62 is fixed to or integrated with protective covering 40. For some applications, protective covering 40 has no holes, and the desiccant is on the face of protective covering 40 that faces the drug. For other applications, protective covering 40 has holes, and the desiccant is on the face of protective covering 40 that is away from the drug, whereby the holes provide fluid communication between the desiccant and the drug.

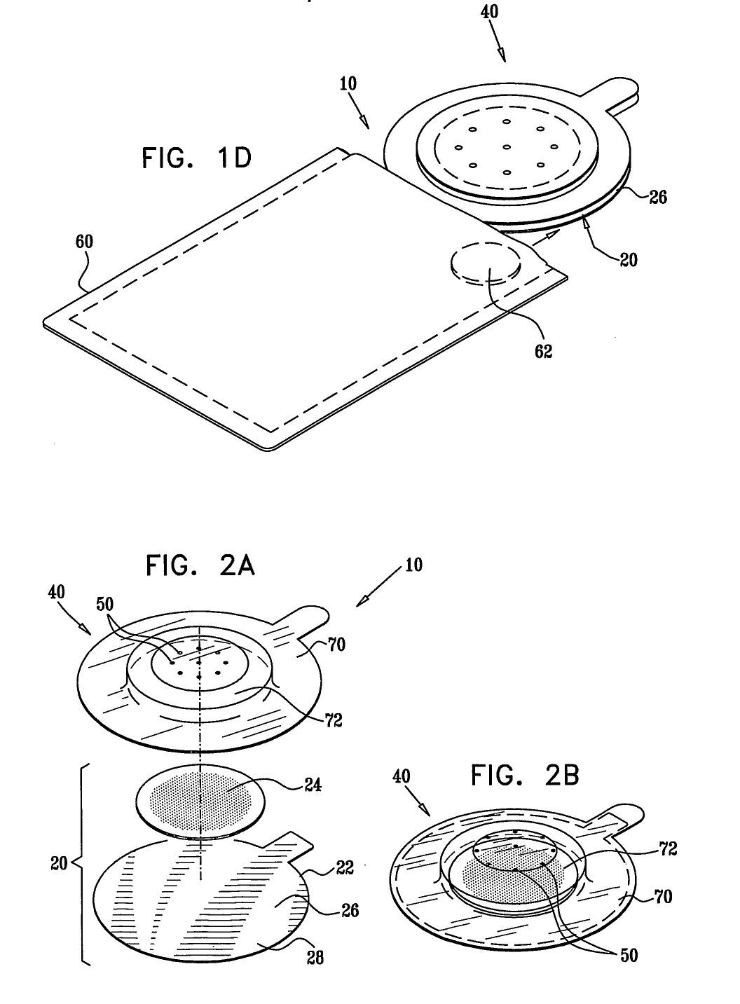

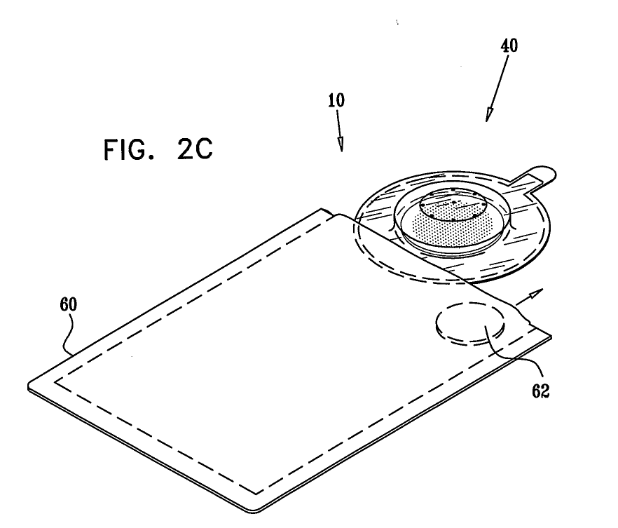

Reference is made to FIGS. 2A-C, which are schematic illustrations of another embodiment of transdermal drug delivery product 10. Except as described below, this embodiment is the same as the embodiment described hereinabove with reference to FIGS. 1A-D. In this embodiment, protective covering 40 comprises a single element, which is shaped so as to define: (a) a peripheral area 70, which is brought into contact with adhesive area 30 of backing liner 22, and (b) a raised central area 72, which provides protection for drug delivery area 24 from mechanical contact with the environment, and is shaped so as not to come into contact with drug delivery area 24, or so as to minimize the likelihood of such contact under normal handling conditions. Raised central area 72 of protective covering 40 is typically configured to allow gas passage therethrough. Typically, raised central area 72 is shaped so as to define one or more holes 50 therethrough for this purpose.

For some applications, protective covering 40 comprises a thermoformable film (e.g., PVC, PETG, or HDPE), which is coated on one side, such as siliconized, and then formed to the desired blister shape using a vacuum-forming process. The film typically has a thickness of between about 150 and about 300 microns. For applications in which adhesive area 30 comprises an acrylic pressure-sensitive adhesive (PSA), the silicone coating provides a release peeling force with the adhesive layer of between about 10 and 30 g per two-inch strip when pulled at 180 degrees, and thus enables easy release while providing good protection to adhesive area 30 and drug delivery area 24. Alternatively, protective covering 40 is manufactured by forming an uncoated film to the desired blister shape, and then laminating or coating the blister rim (the lower side) with a silicone layer.

Reference is made to FIGS. 1A-D and 2A-C. In an embodiment of the present invention, during manufacture of product 10, a drug in a liquid form is applied to drug delivery area 24, such as by using a process like that which is used in ink-jet printing. For some applications, the drug is in a suspension and/or solution when it is applied to area 24. For some applications, the drug is applied as a plurality of small droplets on drug delivery area 24 by a syringe that moves over the surface of area 24 or stays stationary while area 24 moves thereunder. Alternatively or additionally, the drug is applied to area 24 using a technique that is known in the art, such as spraying, rolling, or coating the drug onto area 24. For greater stability and shelf life of the drug, it is desirable that the drug be sufficiently dry during storage of product 10. However, many drugs dry slowly after application to the drug delivery area (for example, some drugs do not dry sufficiently for long-term stability of the drug until up to about 1-4 weeks after application to the drug delivery area). Waiting for the drug to dry sufficiently for long-term stability generally increases manufacturing cost and complexity. Use of protective covering 40 and package 60, as described hereinabove, allows patch 20 to be packaged immediately upon application of the drug, without waiting for the drug to dry, or within about 30-120 minutes (e.g., 30-60 minutes) following application of the drug, while the drug is only partially dry. While packaged, the drug is in fluid (i.e., gaseous) communication, via holes 50, with desiccant 62, which absorbs moisture from the drug, thereby drying the drug to a level sufficient for long-term storage (typically within about 1-4 weeks of packaging). At the same time, upper layer 44 of protective covering 40 protects the drug from mechanical contact with the environment, including the inside of package 60.

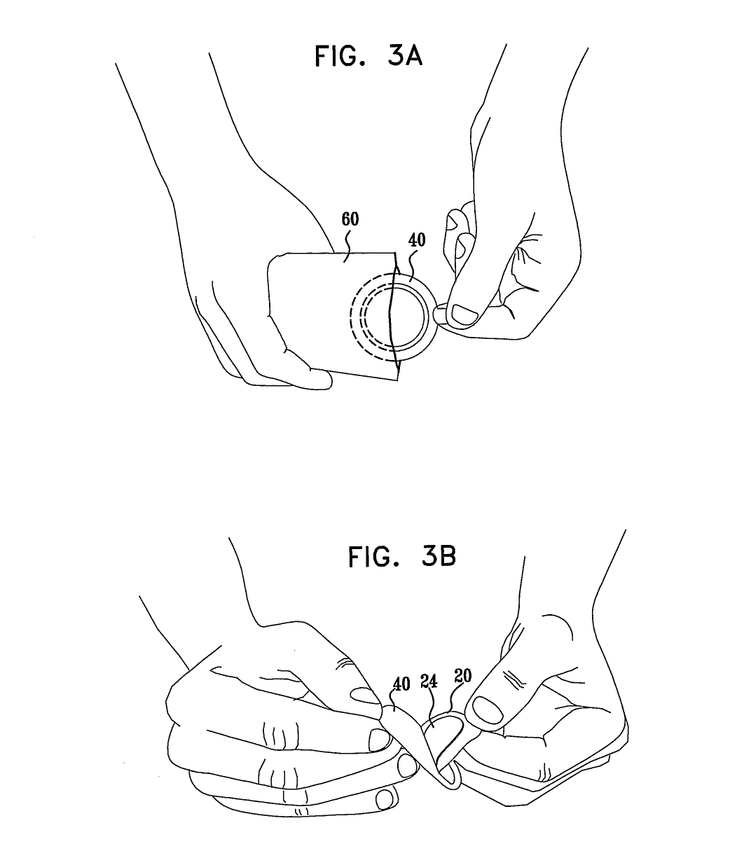

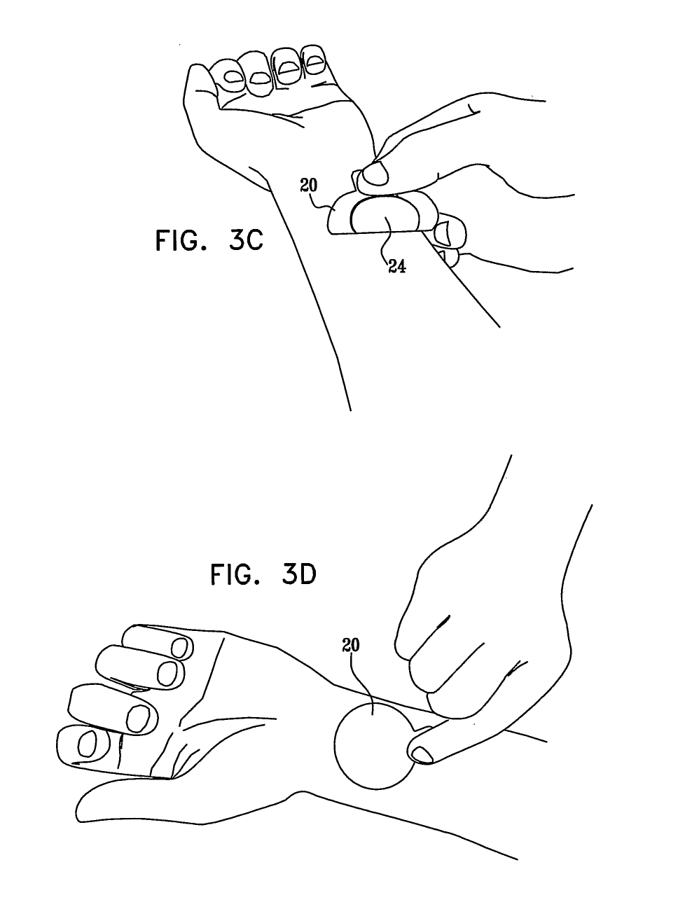

Reference is made to FIGS. 3A-D, which are schematic illustrations of application of patch 20 to skin of a subject, in accordance with an embodiment of the present invention. FIG. 3A shows the subject removing patch 20, together with protective covering 40, from package 60. The subject then peels patch 20 apart from protective covering 40, as shown in FIG. 3B. The subject applies the patch to skin, as shown in FIGS. 3C and 3D. In an embodiment, drug delivery area 24 protrudes about 1-3 or about 3-5 mm from patch 20 (FIGS. 1A, 3B, and 3C), such that the application of patch 20 to the skin enhances the contact pressure between drug delivery area 24 and the skin.

In an embodiment, transdermal drug delivery product 10 is adapted to be used without any particular skin-preparation measures in advance of placing patch 20 on the skin.

In an embodiment of the present invention, transdermal drug delivery product 10 is adapted to be used in conjunction with a system for increasing the permeability of the skin to the drug stored in patch 20, such as by forming microchannels through at least a portion of the skin, and/or by performing iontophoresis and/or by laser ablation and/or by microneedles and/or by sonophoresis and/or by other techniques known in the art. For example, product 10 may be used in conjunction with the ViaDerm drug delivery system, developed by TransPharma Medical Ltd. (Lod, Israel), aspects of which are described in U.S. Pat. Nos. 6,148,232 and 6,611,706, both of which are assigned to the assignee of the present application and are incorporated herein by reference.

In an embodiment of the present invention, moisture of the subject's body comes in contact with the dry drug stored in patch 20, and dissolves the drug to form a saturated solution or suspension. For example, the moisture may be moisture of the skin of the subject. In embodiments of the present invention in which patch 20 is used in conjunction with a device for forming microchannels through the skin, or otherwise ablating or pre-treating the skin, the moisture may be extracellular fluid of the subject released by the body through the microchannels or other openings in the skin.

Although elements of transdermal drug delivery product 10 are shown in the figures as generally circular in shape, this is for illustrative purposes only. For some applications, such elements are elliptical, rectangular, square, or another shape.

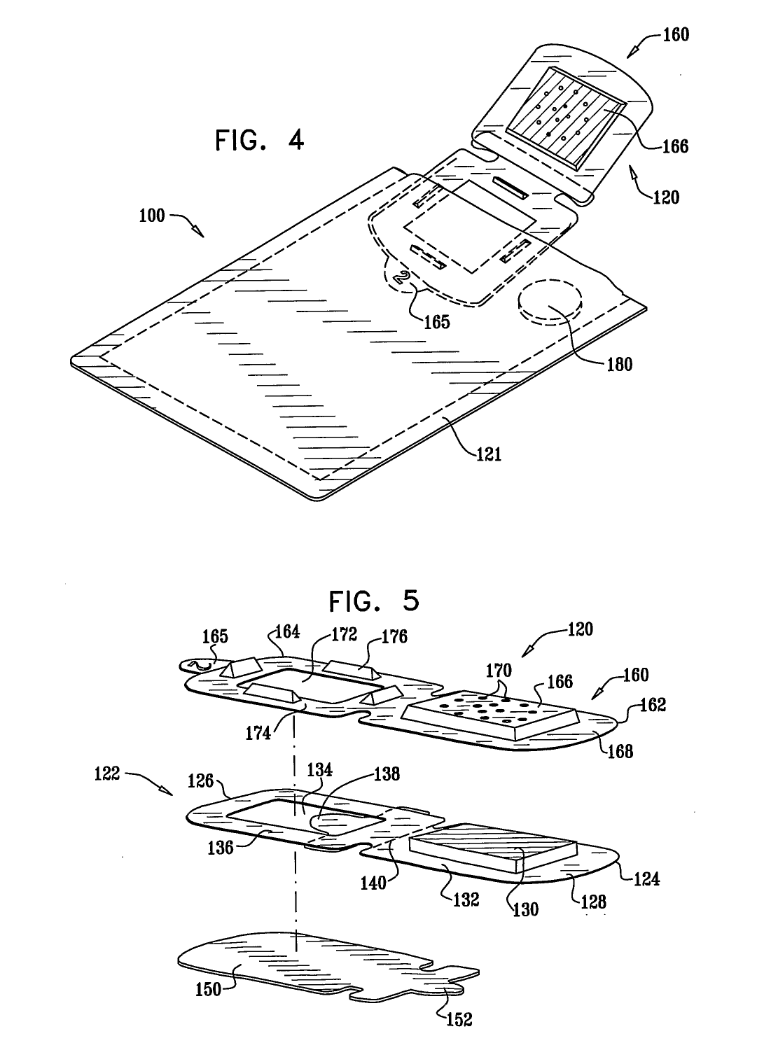

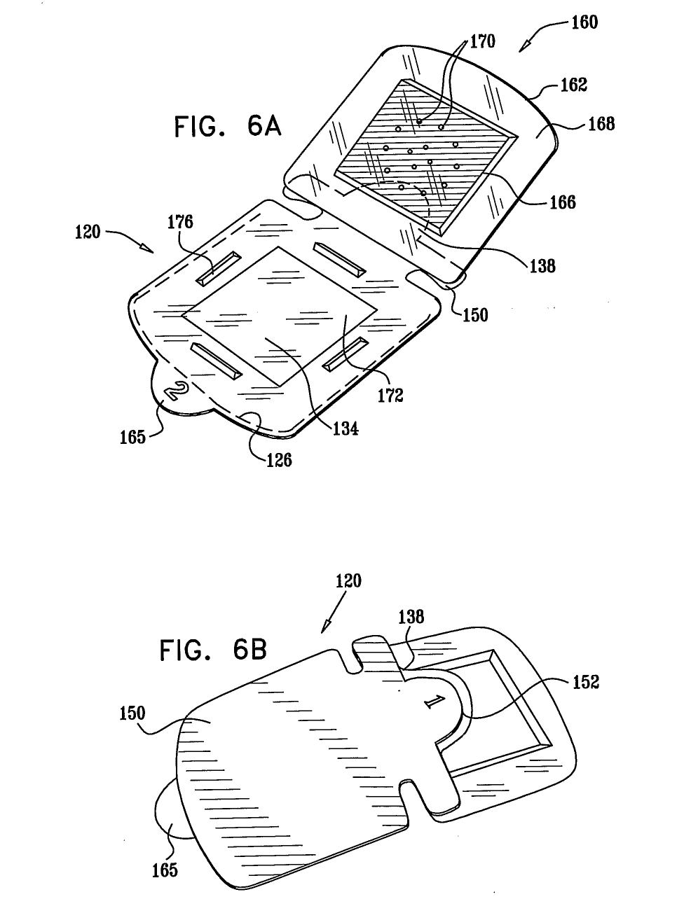

FIGS. 4, 5, and 6A-B are schematic illustrations of a transdermal drug delivery product 100, in accordance with an embodiment of the present invention. As shown in FIG. 4, product 100 comprises a patch 120 and a package 121 for storing patch 120 together with its protective covering. FIG. 5 shows the elements of patch 120 prior to assembly thereof, and FIGS. 6A and 6B are top and bottom views, respectively, of assembled patch 120.

As can best be seen in FIG. 5, patch 120 comprises an elongated support structure 122, which is shaped so as to define a drug support area 124 and a window area 126. An upper surface 128 of drug support area 124 comprises an adhesive, which serves to: (a) adhere a drug delivery area 130, such as a medicated pad, to drug support area 124, and (b) provide an adhesive border 132, the purpose of which is described below. The scope of the invention includes functionally equivalent alternative configurations of patch 120, such as forming drug delivery area 130 as an integrated component of upper surface 128, e.g., by applying a drug directly to a portion of upper surface 128.

Window area 126 is shaped so as to define a window 134 surrounded by a window frame 136, which comprises an adhesive on both sides thereof. Although window 134 is shown as being square in the figures, the window may also be other shapes, such as rectangular, elliptical, or circular, in which case drug delivery area 130 also typically is a corresponding other shape. Support structure 122 is shaped so as to define a first tab 138, which, prior to assembly, as shown in FIG. 5, may protrude into a portion of window 134. Support structure 122 is configured to have a flexible fold line 140 which separates drug support area 124 from window area 126. For some applications, drug support area 124 and window area 124 are formed from separate pieces, respective portions of which are fixed together to form first tab 138 and join the drug support and window areas together along fold line 140.

Patch 120 further comprises a first liner 150, which is configured to be removably coupled to a lower surface of window frame 136 (which, as mentioned above, comprises an adhesive). During assembly of patch 120, prior to coupling the first liner to the window frame, first tab 138 is folded down and towards drug support area 124 (i.e., to the right in the figure), such that a second tab 152 of first liner 150 partially covers first tab 138, without adhering thereto (such partial covering can best be seen in FIG. 6B). For some applications, first liner 150 comprises a PE/PVC substrate having a thickness of between about 0.07 and about 0.10 mm (about 3 to about 4 mils). Typically, first liner 150 is coated with an easy-release coating, such as an easy-release silicone coating.

Patch 120 still further comprises a second liner 160, which is shaped so as to define a protective area 162, a window area 164, and a third tab 165, which protrudes from window area 164 on the side thereof opposite protective area 162. Protective area 162 is shaped so as to define a raised protective area 166 and a border 168. Raised protective area 166 is configured, upon assembly of the patch, to cover drug delivery area 130 while substantially not coming in contact therewith. Border 168, upon assembly of the patch, is removably coupled to adhesive border 132 of drug support area 124 of support structure 122.

Raised protective area 166 is typically configured to allow gas passage therethrough. Typically, raised protective area 166 is shaped so as to define one or more holes 170 therethrough for this purpose. Package 121 contains a gas, which is typically inert, and may comprise, as appropriate, nitrogen and/or argon. Alternatively, the gas comprises air or another combination of gases.

Window area 164 of second liner 160 is shaped so as to define a window 172 surrounded by a window frame 174, which have approximately the same dimensions as window 134 and window frame 136, respectively, of window area 126 of support structure 122. Upon assembly of patch 120, a lower surface of window frame 174 is removably coupled to the adhesive upper surface of window frame 136, and windows 172 and 134 are generally aligned to define a common window through window areas 164 and 126.

For some applications, second liner 160 comprises a PVC substrate having a thickness of between about 0.1 and about 0.2 mm (about 4 to about 8 mils). Typically, second liner is coated with a moderate-release coating, such as a moderate-release silicone coating. For some applications, window frame 174 is shaped so as to define one or more ridges 176 protruding from the upper surface of the window frame.

Reference is again made to FIG. 4. Package 121 typically comprises a moisture-absorbing desiccant 180, such as a silica gel desiccant. For example, desiccant 180 may comprise a conventional desiccant bag or other desiccant container typically used in drug packaging. For some applications, about 0.25 g to about 3 g of desiccant 180 is provided (e.g., about 0.5 g to about 2 g). Desiccant 180 may be fixed to or removable from package 121, as appropriate. Alternatively or additionally, desiccant 180 is fixed to or integrated with raised protective area 166. For some applications, raised protective area 166 has no holes, and the desiccant is on the face of raised protective area 166 that faces the drug. For other applications, raised protective area 166 has holes, and the desiccant is on the face of raised protective area 166 that is away from the drug, whereby the holes provide fluid communication between the desiccant and the drug. As described hereinabove with respect to protective covering 40 and package 60 of product 10, use of raised protective area 166 allows patch 120 to be packaged immediately upon application of the drug, without waiting for the drug to dry, or within about 30-120 minutes (e.g., 30-60 minutes) following application of the drug, while the drug is only partially dry.

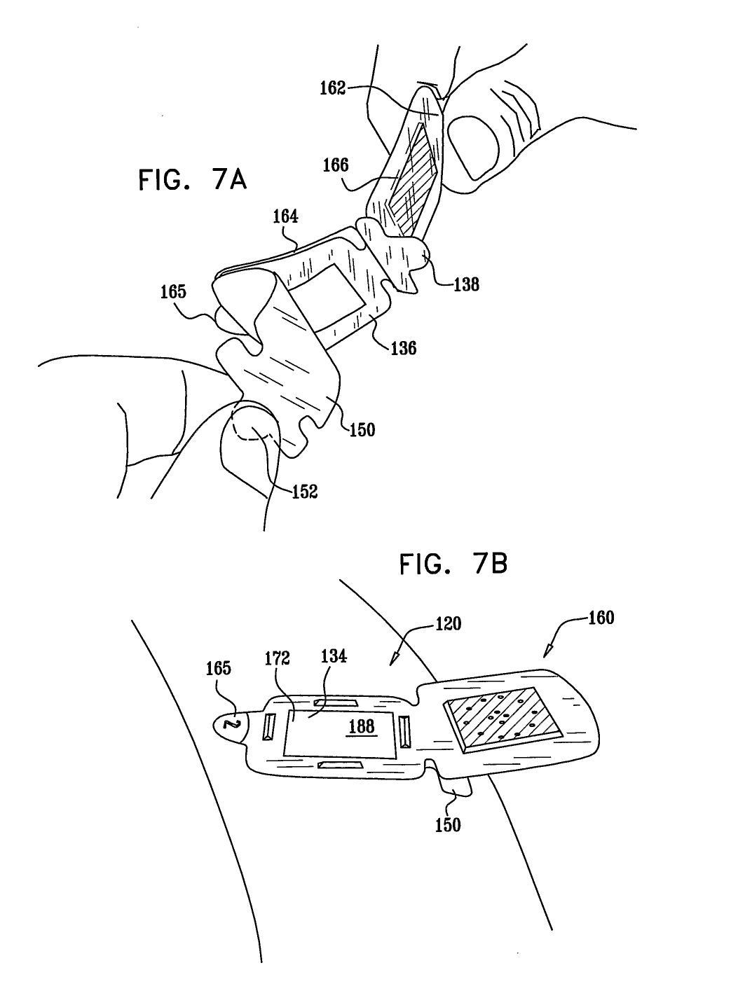

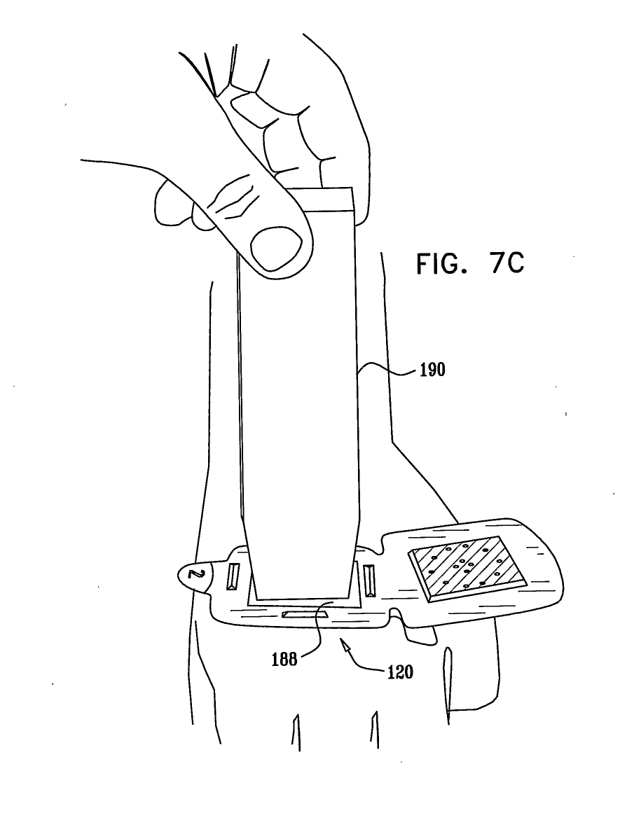

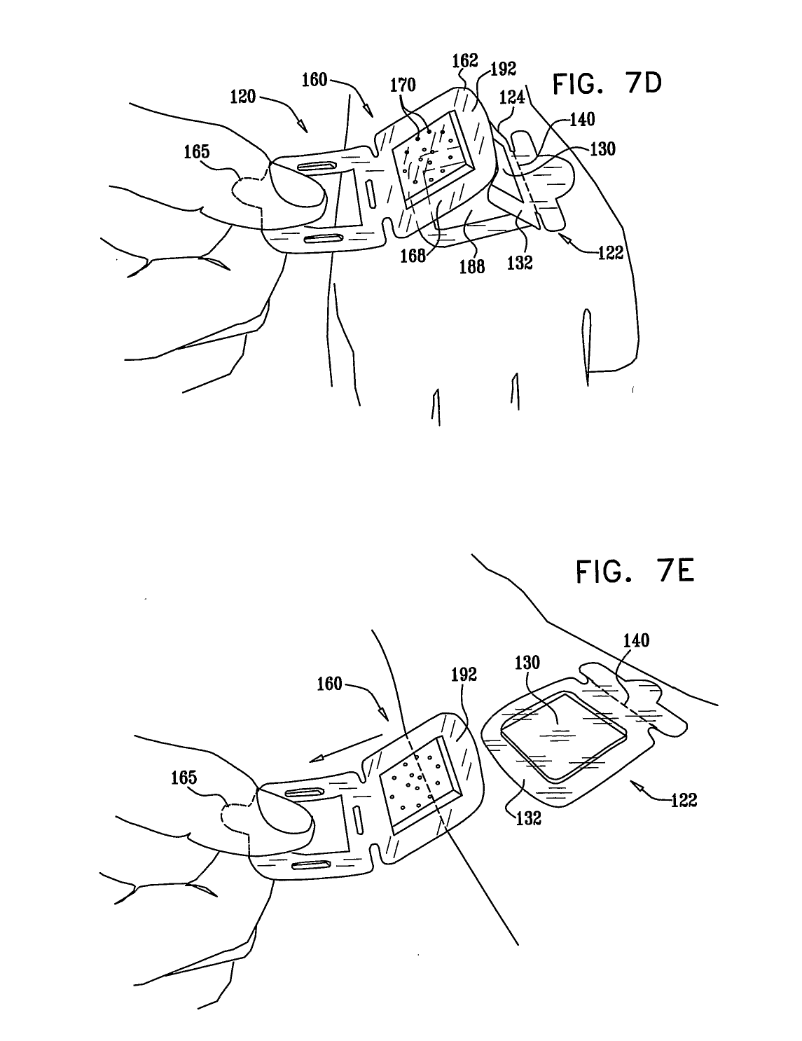

Reference is made to FIGS. 7A-E, which are schematic illustrations of application of patch 120 to skin of a subject, in accordance with an embodiment of the present invention. After removing patch 120, together with its protective covering, from package 121 (removal not shown), the subject peels first liner 150 from window frame 136, by grasping second tab 152 of the liner, as shown in FIG. 7A.

As shown in FIG. 7B, the subject applies the patch to skin, such that a portion 188 of the skin is exposed through windows 134 and 172. The adhesive on the lower side of window frame 136 causes the patch to adhere to the skin. For some applications, the skin is cleaned and dried prior to application of the patch. At this point, drug delivery area 130 is facing away from the skin, and is still covered by raised protective area 166. The lower surface of drug support area 124 rests against the skin (with first tab 138 partially intervening), without adhering to the skin.

As shown in FIG. 7C, the subject applies an applicator 190 to skin portion 188. Windows 134 and 172 thus serve to guide the user to apply the applicator to an area of the skin precisely located with respect to the other elements of patch 120. Applicator 190 comprises a system for increasing the permeability of the skin to the drug stored in patch 120, such as by forming microchannels through at least a portion of the skin, and/or by performing iontophoresis and/or by laser ablation and/or by microneedles and/or by sonophoresis and/or by other techniques known in the art. For example, applicator 190 may comprise the above-mentioned ViaDerm drug delivery system, aspects of which are described in the above-mentioned U.S. Pat. Nos. 6,148,232 and 6,611,706.

After skin portion 188 has been permeability-enhanced, the subject grasps third tab 165, and pulls second liner 160 towards and over skin portion 188, as shown in FIG. 7D. Such pulling causes three sides of border 168 of protective area 162 to become detached from adhesive border 132 of drug support area 124, while leaving a remaining side 192 of border 168 coupled to the adhesive border.

The subject continues to pull second liner 160, until drug delivery area 130 is completely inverted and is brought in contact with skin portion 188, and side 192 of border 168 becomes detached from adhesive border 132, as shown in FIG. 7E. Support structure 122 is now completed folded along fold line 140, and is adhered to the skin around skin portion 188 by adhesive border 132.

In an embodiment of the present invention, moisture of the subject's body comes in contact with the dry drug stored in patch 120, and dissolves the drug to form a saturated solution or suspension. For example, the moisture may be moisture of the skin of the subject, or extracellular fluid of the subject released by the body through the microchannels or other openings in the skin created by applicator 190.

Although elements of transdermal drug delivery product 100 are shown in the figures as generally rectangular (e.g., square) in shape, this is for illustrative purposes only. For some applications, such elements are elliptical (e.g., circular), or other shapes.

It is noted that, by way of illustration and not limitation, the figures show a relatively small number of holes 50 and 170 that are relatively large. Such configurations typically have between about 3 and 50 or between about 50 and 200 holes that are between about 0.5 and 1 mm or between about 1 and 3 mm in diameter. In other embodiments (not shown), the holes are smaller, e.g., between about 0.01 and 0.1 mm or between about 0.1 and 0.5 mm in diameter. Alternatively or additionally, the number of holes is larger, e.g., between about 200 and 1000, or more than 1000. For example, upper layer 44 (FIG. 1A) or second liner 160 (FIGS. 4-6B) may comprise a material such as a cloth, plastic, screen, mesh, porous material, and/or moisture-permeable film, through which a very large number of holes are created or naturally exist. As appropriate, the material may be held taut and/or it may be supported by supporting structures so as to minimize contact between upper layer 44 or raised protective area 166 and the drug.

In an embodiment, techniques and apparatus described herein are combined with techniques and apparatus described in International Application PCT/IL02/00896, filed Nov. 7, 2002, which is assigned to the assignee of the present application and is incorporated herein by reference.

It will be appreciated by persons skilled in the art that the present invention is not limited to what has been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description.

1. Apparatus comprising a transdermal drug delivery patch product, which comprises:
a patch, which comprises a drug; and
protective packaging, adapted to store the patch, and to allow the drug to dry while the patch is stored in the protective packaging.

2. The apparatus according to claim 1, wherein the protective packaging is configured to dry the drug to a moisture content of 2-3% by weight while the patch is stored in the protective packaging.

3. The apparatus according to claim 1, wherein the protective packaging is configured to dry the drug to a moisture content of 3-5% by weight while the patch is stored in the protective packaging.

4. The apparatus according to claim 1, wherein the patch is shaped so as to define:
a first portion shaped so as to define a frame that surrounds a window, and
a second portion, adjacent to the first portion, that defines a drug delivery area comprising the drug, and
wherein the patch is configured such that when the second portion is folded onto the first portion, the drug delivery area is placed through the window.

5. The apparatus according to claim 1, wherein the protective packaging comprises a package, adapted to store the patch, and wherein the package comprises a desiccant in fluid communication with the drug when the patch is stored in the package.

6. The apparatus according to claim 1, wherein the protective packaging comprises a removable protective covering applied to the patch, configured such that during storage, when the protective covering is applied to the patch, at least a portion of the protective covering is spaced away from the drug, and wherein the protective covering is shaped so as to define one or more holes therethrough.

7. The apparatus according to claim 6, wherein the protective packaging comprises a package, adapted to store the patch together with the protective covering, and comprising a desiccant in fluid communication with the drug via the holes when the patch is stored in the package.

8. The apparatus according to claim 6, wherein the protective covering comprises exactly one element.

9. The apparatus according to claim 8, wherein the element is shaped so as to define a peripheral area, and a raised central area shaped so as not to come in contact with the drag.

10. The apparatus according to claim 1, wherein the protective packaging comprises a removable protective covering applied to the patch, configured such that during storage, when the protective covering is applied to the patch, at least a portion of the protective covering is spaced away from the drag, and wherein the protective covering comprises a desiccant.

11. The apparatus according to claim 10, wherein the desiccant is disposed on a face of the protective covering that faces the patch.

12. The apparatus according to claim 10, wherein the protective covering is shaped so as to define one or more holes therethrough, and wherein the desiccant is disposed on a face of the protective covering that faces away from the patch, such that the desiccant is in fluid communication with the drag via the holes when the patch is stored in the protective packaging.

13. The apparatus according to claim 6, wherein the protective covering is shaped so as to define between 1 and 10 holes therethrough.

14. The apparatus according to claim 6, wherein the protective covering is shaped so as to define between 10 and 100 holes therethrough.

15. The apparatus according to claim 6, wherein the protective covering is shaped so as to define greater than 100 holes therethrough.

16. The apparatus according to claim 6, wherein the protective covering comprises at least one item selected from the list consisting of: a cloth, a plastic, a screen, a mesh, a porous material, and a moisture-permeable film, and wherein the one or more holes are holes in the selected item.

17. A method for manufacturing a transdermal drag delivery patch product, the method comprising:
applying a drag to a patch; and
while at least a portion of the drug has greater than 6% moisture content by weight, storing the patch in protective packaging adapted to dry the drug while the patch is stored in the protective packaging.

18. The method according to claim 17, wherein storing the patch in the protective packaging comprises placing a desiccant in the protective packaging.

19. The method according to claim 17, wherein storing the patch comprises storing the patch in the protective packaging, the protective packaging being adapted to dry the drug to a moisture content of 2-3% by weight while the patch is stored in the protective packaging.

20. The method according to claim 17, wherein storing the patch comprises storing the patch in the protective packaging, the protective packaging being adapted to dry the drug to a moisture content of 3-5% by weight while the patch is stored in the protective packaging.

21. The method according to claim 17, wherein the protective packaging includes a package which includes a desiccant, and wherein storing the patch comprises storing the patch in the package such that the desiccant is in fluid communication with the drug.

22. The method according to claim 17, wherein the protective packaging includes a removable protective covering shaped so as to define one or more holes therethrough, and wherein storing the patch comprises applying the protective covering to the patch such that at least a portion of the protective covering is spaced away from the drug.

23. The method according to claim 22, wherein the protective packaging includes a package which includes a desiccant, and wherein storing the patch comprises storing the patch in the package together with the protective covering such that the desiccant is in fluid communication with the drug via the holes.

24. The method according to claim 17, wherein the protective packaging includes a removable protective covering including a desiccant, and wherein storing the patch comprises applying the protective covering to the patch such that at least a portion of the protective covering is spaced away from the drug.

25. The method according to claim 24, wherein the desiccant is disposed on a face of the protective covering that faces the patch.

26. The method according to claim 24, wherein the protective covering is shaped so as to define one or more holes therethrough, wherein the desiccant is disposed on a face of the protective covering that faces away from the patch, and wherein storing the patch comprises applying the protective covering to the patch such that the desiccant is in fluid communication with the drug via the holes.

27. A method for transdermally administering a drug to a subject, comprising:
applying a drug to a patch;
while at least a portion of the drug has greater than 6% moisture content by weight, storing the patch in protective packaging;
allowing the drug to dry while the patch is stored in the package; and
after the drug dries, applying the patch to skin of the subject, such that moisture from the skin dissolves the drug.

28. The method according to claim 27, wherein the patch is shaped so as define a first portion and a second portion adjacent to the first portion, and wherein applying the patch to the skin comprises:
adhering a frame of the first portion to the skin such that a portion of the skin is exposed through a window defined by the frame; and
folding the second portion onto the first portion, such that a drug delivery area of the second portion that comprises the drug is applied through the window to the portion of the skin.

29. The method according to claim 27, wherein storing the patch in the protective packaging comprises placing a desiccant in the protective packaging.

30. The method according to any one of claims 27 29claim 27, wherein the protective packaging includes a removable protective covering shaped so as to define one or more holes therethrough, wherein storing the patch comprises applying the protective covering to the patch such that at least a portion of the protective covering is spaced away from the drug, and wherein applying the patch comprises removing the protective covering from the patch.

31. The method according to claim 30, wherein the protective packaging includes a package which includes a desiccant, and wherein storing the patch comprises storing the patch in the package together with the protective covering such that the desiccant is in fluid communication with the drug via the holes.
